(19) United States
(12) Patent Application Publication
(10) Pub. No.: US 2024/0350375 A1
(43) Pub. Date: Oct. 24, 2024

(54) NON-NANO UV FILTER DISPERSIONS

(71) Applicant: BASF SE, Ludwigshafen am Rhein (DE)

(72) Inventors: Ullrich MENGE, Grenzach-Wyhlen (DE); Frank MÜLLER, Ludwigshafen am Rhein (DE)

(21) Appl. No.: 18/687,319

(22) PCT Filed: Aug. 11, 2022
(86) PCT No.: PCT/EP2022/072536

§ 371 Date: Feb. 28, 2024

(30) Foreign Application Priority Data
Aug. 31, 2021 (EP) 21193897.2

(51) Int. Cl. A61K 8/06; A61Q 17/04

(52) U.S. Cl. CPC A61K 8/068; A61Q 17/04; A61K 2800/412; A61K 2800/805

(57) ABSTRACT

The present invention relates to a process of manufacturing an aqueous suspension (1) of at least one micronized organic UV filter as well as to an aqueous suspension (1) comprising at least one micronized organic UV filter.

The present invention relates to a process of manufacturing an aqueous suspension (1) of at least one micronized organic UV filter as well as to an aqueous suspension (1) comprising at least one micronized organic UV filter and the use thereof.

Micronization of organic UV filter (also known as UV absorber) dispersions is known in the literature.

Suitable organic particulate UV absorbers for use in cosmetic sunscreens are described e.g. in WO9703643, WO2009077356 and WO2015155158.

WO9703643 provides a method for producing a composition of a micronized organic UV absorber, which comprises the grinding the UV absorber in the presence of an alkyl polyglucoside. The mean particle size of the micronized organic UV Filter is 0.01-2 μm, especially from 0.05-1 μm.

WO2009003934 describes further methods for producing new nanoscalar formulations of micronized, insoluble UV absorbers. The dispersions are prepared by grinding the UV absorber in an apparatus comprising yttrium-stabilized zirconium oxide grinding beads in the presence of an antifoam agent as dispersing agent auxiliary.

With respect to use of antifoam in a grinding process, WO2018069200 claims that the foaming is significantly reduced if a specific particle size of the coarse UV absorber is selected. Specifically, to achieve a particle size Dv50<200 nm (determined by light scattering) of a dispersion of UV absorber in a mixture of water and alkyl polyglucoside, it is required that the Dv90 of the particle size distribution as determined by laser diffraction of the UV absorber solid is within 1-150 μm.

Grinding aids for use in the production of micronized organic UV absorbers are exemplarily described in WO2009068469.

None of these publication teaches how to prepare non-nano UV absorber suspension, which still provide efficient UV protection. In view of the ongoing public discussion on use of nano materials e.g. in cosmetic products, there is an ongoing need to develop a process for the production of UV absorber suspensions that are no nano material.

The particle size distribution of a suspension may be characterized with respect to particle volume (mass), or to the number of particles. For instance, Dv90=1 μm means that 90% of the volume (or mass) of the dispersed material consists of particles smaller than 1 μm, and 10% are larger. In contrast, D_{N}30=100 nm refers to the number-based size distribution and indicates that 30% of all particles in the sample are smaller than 100 nm. As a rule of the thumb, in a polydisperse sample the small particles dominate number-based distributions D_{N} while large particles dominate Dv. Measurement techniques differ in their sensitivity towards particle number or particle mass. Volume based distributions may be obtained by laser diffraction, and number-based distributions by electron microscopy. As for laser diffraction, many commercial instruments are available, for example from Anton Paar (PSA series), Microtrac MRB (Sync) or Malvern Panalytical (Mastersizer series). Depending on sensitivity and resolution of the selected instrument, the numerical results characterizing the particle size distribution will differ within some minor range. The skilled expert knows how to deal with these deviations, and such instruments are routinely used in R&D and quality control labs. In contrast, electron microscopy is more expensive and less frequently used.

This distinction between D_{N} and Dv is relevant for the “nano” classification of materials. In accordance with the recommendation of the European Commission 2011/696/EU, a micronized dispersion with a D_{N}50 value of 100 nm or below is a nano material. The methods found in the literature for the production of micronized organic UV absorbers usually end up in nano dispersions, i.e. comprising UV absorber particles Dv50 of the nano-sized insoluble organic UV absorber in the range of 50 to 150 nm (WO2018069200). This seems desirable as the efficiency of the UV absorber dispersion increases with decreasing particle size. Furthermore, depending on the mechanical properties of the UV absorber crystals, the breaking mechanism in a grinding device may generate nano material out of crystals that are still in the μm range.

Against the above-outlined background it was an object of the present invention to provide a formulation comprising micronized, non-nano-sized organic UV filter, preferably wherein the micronized, non-nano-sized organic UV filter is an insoluble organic UV filter. Further, it was an object of the present invention that the non-nano-sized organic UV filter still provides sufficient UV protection efficiency. It was further an object of the present invention to provide a process of manufacturing a formulation comprising micronized, non-nano-sized organic UV filter, preferably wherein the micronized, non-nano-sized organic UV filter is an insoluble organic UV filter. It was a further object of the present invention to provide a formulation comprising no nano material.

Surprisingly, it has been found that if a hydrophobic additive is added in the grinding process at an adjusted temperature in the mill, the formation of nano material in the micronization process is avoided, whereby a sufficient efficiency of the UV filter can be remained.

Hence, according to a first aspect A, the present invention relates to a process of manufacturing an aqueous suspension (1) of at least one organic UV filter having a particle size D_{N}30 of 100 nm or more, said process comprising the step of milling a suspension (2) comprising the at least one organic UV filter in a mixture of water and a hydrophobic additive in a milling apparatus at a temperature of 35 to 90° C.

In view of the EC Nano recommendation, it may be noted that in principle the invention can also be used to achieve an aqueous suspension (1) of at least one organic UV filter having a particle size within D_{N}30 of 100 nm and D_{N}50 of 100 nm. In view of fluctuations in the particle size distribution, it is preferred to aim at a particle size which is less close to the Nano material borderline and avoid the analysis of every single batch in real production.

In the following, preferred embodiments of the above process of manufacturing are described in further detail. Further, preferred embodiment of an aqueous suspension (1) and the use thereof are described in more detail. It is to be understood that each preferred embodiment is relevant on its own as well as in combination with other preferred embodiments.

In a preferred embodiment A1 of the first aspect, the at least one organic UV filter in the aqueous suspension (1) has a particle size D_{N}10, preferably determined by transmission electron microscopy, of 100 nm or more, preferably of 0.1 to 0.4 μm, more preferably of 0.1 to 0.2 μm; and/or
a particle size D_{N}50, preferably determined by transmission electron microscopy, of 120 nm or more, preferably of from 0.12 to 0.5 μm, more preferably from 0.12 to 0.3 μm; and/or
a particle size D_{N}90, preferably determined by transmission electron microscopy, of 200 nm or more, preferably of from 0.2 to 1.0 μm, more preferably from 0.2 to 0.5 μm; and/or
a particle size Dv10 determined by laser diffraction of more than 0.1 μm, preferably of from 0.1 to 0.4 μm; and/or
a particle size Dv90 determined by laser diffraction of less than 2.2 μm, preferably of less than 2.0 μm, more preferably of less than 1.5 μm,
where Dv10 and Dv90 are measured using a Mastersizer 2000 from Malvern Panalytical

In a preferred embodiment A2 of the first aspect, the population of particles below 100 nm in the aqueous suspension (1) is reduced during the milling step, preferably wherein the population of particles below 100 nm in the aqueous suspension (1) is reduced by increasing the temperature of the suspension (2) in the milling apparatus. The terms “is reduced” and “increasing the temperature” both relate to the comparison with a milling step that yields an aqueous suspension (1) with a D_{N}30 of 99 nm or less.

In a preferred embodiment A3 of the first aspect, the temperature of the suspension (2) in the milling step is in the range of 40 to 80° C., preferably of 40 to 70° C., and in particular of 45 to 65° C.

In a preferred embodiment A4 of the first aspect, the hydrophobic additive is a cosmetic oil, preferably selected from the group consisting of alcohols having from 6 to 18 carbon atoms; C6-C24 carboxylic acids; esters of C6-C24 carboxylic acids with C3-C24 alcohols; esters of hydroxycarboxylic acids with C6-C24 alcohols; esters of carboxylic acids with polyhydric alcohols; liquid mono-/di-/tri-glyceride mixtures based on C6-C18 carboxylic acids; esters of C6-C24 alcohols with aromatic carboxylic acids or with oxo carboxylic acids; tricarboxylic acid esters; esters of C2-C12 dicarboxylic acids with alcohols having from 1 to 22 carbon atoms or polyols having from 2 to 10 carbon atoms and from 2 to 6 hydroxy groups; substituted cyclohexanes; C6-C22 alcohol carbonates; symmetric or asymmetric dialkyl ethers having a total of from 12 to 36 carbon atoms; ring-opening products of epoxidized carboxylic acid esters with polyols; silicone oils; aliphatic or naphthenic hydrocarbons; diol esters; and mixtures thereof, in particular selected from the group consisting of esters of C6-C24 alcohols, preferably C10 to C17 alcohols, with aromatic carboxylic acids, preferably benzoic acid, or with hydroxycarboxylic acids, preferably lactate, or with oxo carboxylic acids, preferably levulinate; dicarboxylic acid esters, preferably di-n-butyl adipate; tricarboxylic acid esters, preferably tributyl citrate; and mixtures thereof.

In a preferred embodiment A5 of the first aspect, the at least one organic UV filter is an insoluble organic UV filter, preferably selected from the group consisting of oxanilide UV filter, triazine UV filter, piperazine UV filter, triazole UV filter, vinyl group-containing amide UV filter, cinnamic acid amide UV filter, sulfonated benzimidazole, and mixtures thereof, more preferably selected from the group consisting of oxanilide UV filter having the formula (1)

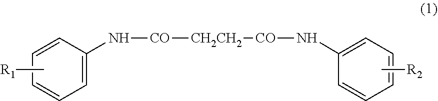

in which R₁ and R₂ are independently C1-C18 alkyl or C1-C18 alkoxy; triazine UV filter having the formula (2)

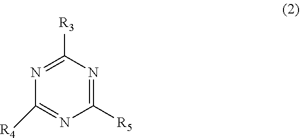

in which R₃, R₄ and R₅, independently, are H; OH; C1-C18 alkoxy; NH2; NH—R6 or N(R6)2 in which R6 is C1-C18 alkyl; OR6 in which R6 has its previous significance; phenyl; phenoxy; anilino; pyrrolo, in which the respective phenyl, phenoxy, anilino, or pyrrolo moieties are optionally substituted by one, two or three substituents selected from OH, carboxy, CO—NH2, C1-C18 alkyl or -alkoxy, C1-C18 carboxyalkyl, C5-C8 cycloalkyl, phenyl, a methylidenecamphor group, a group —(CH═CH)mC(═O)—OR6 in which m is 0 or 1 and R6 has its previous significance, or
a group

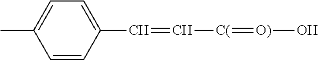

or the corresponding alkali metal, ammonium, mono-, di- or tri-C1-C4 alkylammonium, mono-, di- or tri-C2-C4 alkanolammonium salts, or the C1-C18alkyl esters thereof; phenylene bis-diphenyltriazine; a piperazine UV filter; a triazole UV filter having the formulae (31), (32) or (33)

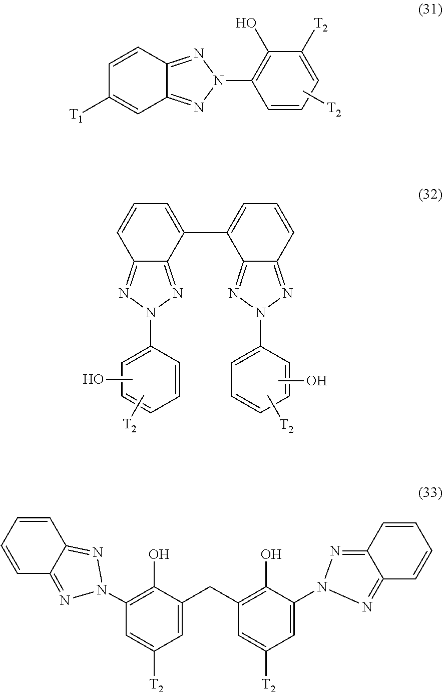

in which T₁ is C1-C18 alkyl or hydrogen and T₂ is C1-C18-alkyl, which is optionally substituted by phenyl; a vinyl group-containing amide UV filter having the formula (4)

R₉—(Y)ₘ—CO—C(R₁₀)═C(R₁₁)13 N(R₁₂)(R₁₃) (4)

in which R₉ is C1-C18 alkyl or phenyl optionally substituted by one, two or three substituents selected from OH, C1-C18 alkyl, C1-C18 alkoxy or CO—OR6 in which R6 has its previous significance; R₁₀, R₁₁, R₁₂ and R₁₃ are the same or different and each is independently C1-C18 alkyl or hydrogen; Y is N or O; and m has its previous significance; cinnamic acid amide UV filter having the formula (5)

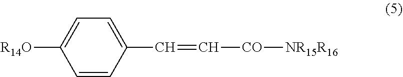

in which R₁₄ is hydroxy or C1-C4 alkoxy; R₁₅ is hydrogen or C1-C4 alkyl; and R₁₆ is —(CONH)m-phenyl in which m has its previous significance and the phenyl group is optionally substituted by one, two or three substituents selected from OH, C1-C18 alkyl, C1-C18 alkoxy or CO—OR6 in which R6 has its previous significance; sulfonated benzimidazole UV filter having the formula (6)

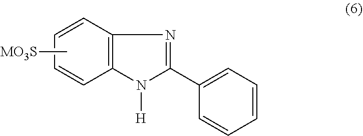

in which M is hydrogen or an alkali metal, an alkaline earth metal or zinc; and mixtures thereof, even more preferably wherein the at least one organic UV filter is selected from the group consisting of tris-biphenyl triazine, 1,1′-(1,4-piperazinediyl)bis[1-[2-[4-(diethylamino)-2-hydroxybenzoyl]phenyl]-methanone, phenylene bis-diphenyltriazine, and 2,2′-methylenebis[6-(2H-1,2,3-benzotriazol-2-yl)-4-(2,4,4-trimethylpentan-2-yl)phenol], and in particular wherein the at least one organic UV filter is selected from the group consisting of tris- biphenyl triazine and 2,2′-methylenebis[6-(2H-1,2,3-benzotriazol-2-yl)-4-(2,4,4-trimethylpentan-2-yl)phenol].

In a preferred embodiment A6 of the first aspect, the hydrophobic additive is comprised in the aqueous suspension (1) in an amount of 0.01 to 10.0 wt.-%, preferably of 0.01 to 5.0 wt.-%, more preferably of 0.01 to 3.0 wt.-%, and in particular of 0.05 to 1.0 wt.-%, based on the total amount of the aqueous suspension (1).

In a preferred embodiment A7 of the first aspect, the milling step is conducted in a ball mill, a vibratory mill, a wet rotor mill, a stirred media mill, or a colloid mill, preferably a wet rotor mill or a stirred media mill, and in particular in a stirred media mill with milling beads, preferably glass beads, zirconium oxide or mixed ceramic grinding beads, having a diameter of 0.1 to 10 mm, preferably of 0.15 to 5 mm, and in particular of 0.2 to 3 mm.

In a preferred embodiment A8 of the first aspect, the at least one organic UV filter in suspension (2) has a particle size Dv90 determined by laser diffraction in the range of from 0.01 to 300 μm, preferably from 0.1 to 250 μm.

In a second aspect B, the present invention relates to aqueous suspension (1) comprising
a) 10 to 65 wt.-% of at least one organic UV filter having a particle size D_{N}30 of 100 nm or more
b) 0.01 to 10.0 wt.-% of a hydrophobic additive selected from the group consisting of alcohols having from 6 to 18 carbon atoms; C6-C24 carboxylic acids; esters of C6-C24 carboxylic acids with C3-C24 alcohols; esters of hydroxycarboxylic acids with C6-C24 alcohols; esters of carboxylic acids with polyhydric alcohols; liquid mono-/di-/tri-glyceride mixtures based on C6-C18 carboxylic acids; esters of C6-C24 alcohols with aromatic carboxylic acids or with oxo carboxylic acids; tricarboxylic acid esters; esters of C2-C12 dicarboxylic acids with alcohols having from 1 to 22 carbon atoms or polyols having from 2 to 10 carbon atoms and from 2 to 6 hydroxy groups; substituted cyclohexanes; C6-C22 alcohol carbonates; symmetric or asymmetric dialkyl ethers having a total of from 12 to 36 carbon atoms; ring-opening products of epoxidized carboxylic acid esters with polyols; silicone oils; aliphatic or naphthenic hydrocarbons; diol esters; and mixtures thereof, and
c) water

each based on the total amount of the aqueous suspension (1).

In a preferred embodiment B1 of the second aspect, the hydrophobic additive is selected from the group consisting of esters of C6-C24 alcohols, preferably C10 to C17 alcohols, with aromatic carboxylic acids, preferably benzoic acid, or with hydroxycarboxylic acids, preferably lactate, or with oxo carboxylic acids, preferably levulinate; dicarboxylic acid esters, preferably di-n-butyl adipate; tricarboxylic acid esters, preferably tributyl citrate; and mixtures thereof. In a preferred embodiment B2 of the second aspect, the at least one organic UV filter is an insoluble organic UV filter, preferably selected from the group consisting of oxanilide UV filter, triazine UV filter, piperazine UV filter, triazole UV filter, vinyl group-containing amide UV filter, cinnamic acid amide UV filter, sulfonated benzimidazole, and mixtures thereof, more preferably selected from the group consisting of oxanilide UV filter having the formula (1)

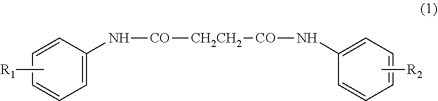

in which R₁ and R₂ are independently C1-C18 alkyl or C1-C18 alkoxy; triazine UV filter having the formula (2)

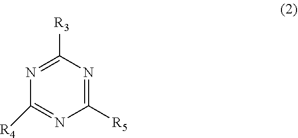

in which R₃, R₄ and R₅, independently, are H; OH; C1-C18 alkoxy; NH2; NH—R6 or N(R6)2 in which R6 is C1-C18 alkyl; OR6 in which R6 has its previous significance; phenyl; phenoxy; anilino; pyrrolo, in which the respective phenyl, phenoxy, anilino, or pyrrolo moieties are optionally substituted by one, two or three substituents selected from OH, carboxy, CO—NH2, C1-C18 alkyl or -alkoxy, C1-C18 carboxyalkyl, C5-C8 cycloalkyl, phenyl, a methylidenecamphor group, a group —(CH═CH)mC(═O)—OR6 in which m is 0 or 1 and R6 has its previous significance, or
a group

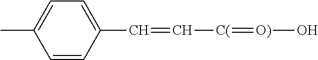

or the corresponding alkali metal, ammonium, mono-, di- or tri-C1-C4 alkylammonium, mono-, di- or tri-C2-C4 alkanolammonium salts, or the C1-C18alkyl esters thereof; phenylene bis-diphenyltriazine; a piperazine UV filter; a triazole UV filter having the formulae (31), (32) or (33)

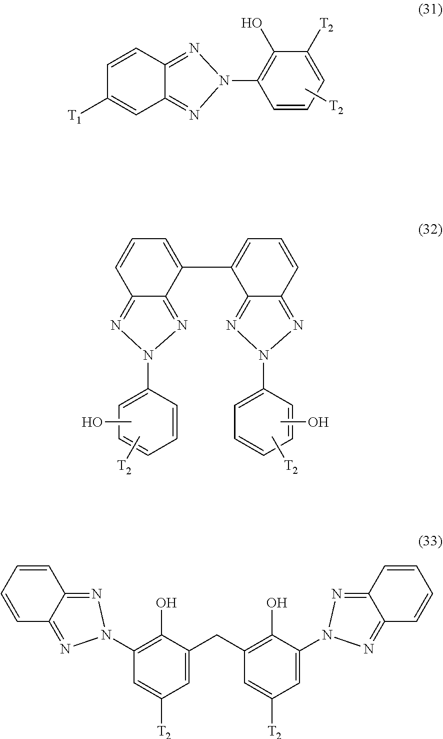

in which T₁ is C1-C18 alkyl or hydrogen and T₂ is C1-C18-alkyl, which is optionally substituted by phenyl; a vinyl group-containing amide UV filter having the formula (4)

R₉—(Y)ₘ—CO—C(R₁₀)═C(R₁₁)—N(R₁₂)(R₁₃) (4)

in which R₁₄ is C1-C18 alkyl or phenyl optionally substituted by one, two or three substituents selected from OH, C1-C18 alkyl, C1-C18 alkoxy or CO—OR6 in which R6 has its previous significance; R₁₀, R₁₁, R₁₂ and R₁₃ are the same or different and each is independently C1-C18 alkyl or hydrogen; Y is N or O; and m has its previous significance; cinnamic acid amide UV filter having the formula (5)

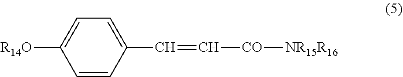

in which R₁₄ is hydroxy or C1-C4 alkoxy; R₁₅ is hydrogen or C1-C4 alkyl; and R₁₆ is —(CONH)m-phenyl in which m has its previous significance and the phenyl group is optionally substituted by one, two or three substituents selected from OH, C1-C18 alkyl, C1-C18 alkoxy or CO—OR6 in which R6 has its previous significance; sulfonated benzimidazole UV filter having the formula (6)

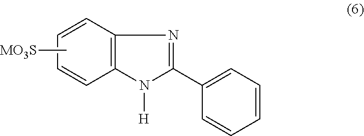

in which M is hydrogen or an alkali metal, an alkaline earth metal or zinc; and mixtures thereof, even more preferably wherein the at least one organic UV filter is selected from the group consisting of tris-biphenyl triazine, 1,1′-(1,4-piperazinediyl)bis[1-[2-[4-(diethylamino)-2-hydroxybenzoyl]phenyl]-methanone, phenylene bis-diphenyltriazine, and 2,2′-methylenebis [6-(2H-1,2,3-benzotriazol-2-yl)-4-(2,4,4-trimethylpentan-2-yl)phenol], and in particular wherein the at least one organic UV filter is selected from the group consisting of tris-biphenyl triazine and 2,2′-methylenebis[6-(2H-1,2,3-benzotriazol-2-yl)-4-(2,4,4-trimethylpentan-2-yl)phenol].

In a preferred embodiment B3 of the second aspect, the aqueous suspension (1) comprises the hydrophobic additive in an amount of 0.01 to 8.0 wt.-%, more preferably of 0.01 to 5.0 wt.-%, more preferably of 0.01 to 3.0 wt.-%, and in particular of 0.05 to 1.0 wt.-%, based on the total amount of the aqueous suspension (1).

In a preferred embodiment B4 of the second aspect, the at least one organic UV filter has a particle size D_{N}10, preferably determined by transmission electron microscopy, of 100 nm or more, preferably of 0.1 to 0.4 μm, more preferably of 0.1 to 0.2 μm; and/or
a particle size D_{N}50, preferably determined by transmission electron microscopy, of 120 nm or more, preferably of from 0.12 to 0.5 μm, more preferably from 0.12 to 0.3 μm; and/or
a particle size D_{N}90, preferably determined by transmission electron microscopy, of 200 nm or more, preferably of from 0.2 to 1.0 μm, more preferably from 0.2 to 0.5 μm; and/or
a particle size Dv10 determined by laser diffraction of more than 0.1 μm, preferably of from 0.1 to 0.4 μm; and/or
a particle size Dv90 determined by laser diffraction of less than 2.2 μm, preferably of less than 2.0 μm, more preferably of less than 1.5 μm,
where Dv10 and Dv90 are measured using a Mastersizer 2000 from Malvern Panalytical

In a third aspect C, the present invention relates to aqueous suspension (1) according to the second aspect B and all its embodiments for use in sunscreen or daily care composition.

### DETAILED DESCRIPTION

Before describing in detail exemplary embodiments of the present invention, definitions which are important for understanding the present invention are given.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As used herein the term “does not comprise” or “free of” means in the context that the composition of the present invention is free of a specific compound or group of compounds, which may be combined under a collective term, that the composition does not comprise said compound or group of compounds in an amount of more than 0.8% by weight, based on the total weight of the composition. Furthermore, it is preferred that the composition according to the present invention does not comprise said compounds or group of compounds in an amount of more than 0.5% by weight, preferably the composition does not comprise said compounds or group of compounds at all.

When referring to compositions and the weight percent of the therein comprised ingredients it is to be understood that according to the present invention the overall amount of ingredients does not exceed 100% (±1% due to rounding).

The term “sunscreen composition” or “sunscreen” refers to any topical product, which absorbs and which may further reflect and scatter certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, foams, powders, or gels. According to the present invention the sunscreen composition may comprise one or more active agents, e.g., organic and inorganic UV filters, as well as other ingredients or additives, e.g., emulsifiers, emollients, viscosity regulators, stabilizers, preservatives, or fragrances.

As suitable inorganic UV filters titanium dioxide, zinc oxide, and cerium oxide may be named.

The term “daily care composition” refers to any topical product, which absorbs and which may further reflect and scatter certain parts of UV radiation and is used as an everyday care product for the human body, e.g. for face or body. The daily care composition may comprise one or more active agents, e.g., organic and/or inorganic UV filters, as well as other ingredients or additives, e.g., emulsifiers, emollients, viscosity regulators, stabilizers, preservatives, or fragrances. Suitable daily care composition are according to the present invention, e.g. leave-on face and body care products.

Suitable leave-on products for face and body are, e.g. sunscreen compositions, decorative preparations, and skin care preparations.

Suitable decorative preparations are, e.g., lipsticks, nail varnishes, eye shadows, mascaras, dry and moist make-up, rouge, powders, depilatory agents and suntan lotions. Suitable skin care preparations are e.g., moisturizing, refining, and lifting preparations. The cited daily care compositions can be in the form of creams, ointments, pastes, foams, gels, lotions, powders, make-ups, sprays, sticks or aerosols. The daily-care compositions are therapeutic daily-care compositions since they comprise the UV filter comprised in the aqueous suspension (1) prepared in the inventive method.

The term “UV filter” or “ultraviolet filter” as used herein refers to organic or inorganic compounds, which can absorb and may further reflect and scatter UV radiation caused by sunlight. UV-filter can be classified based on their UV protection curve as UV-A, UV-B, or broadband filters.

Water soluble UV filters have a solubility in water of at least 2% by weight, preferably at least 3% by weight, more preferably at least 5% by weight.

The prefix Cn-Cm indicates in each case the possible number of carbon atoms in the group.

The term “C₁₂-C₁₅ alkyl benzoate” refers to esters of benzoic acid with fatty alcohols containing a C₁₂-C₁₅-alkyl chain. C₁₂-C₁₅ alkyl chain is defined as an alkyl chain with C₁₂, C₁₃, C₁₄ or C₁₅ chain length.

The term “Cn-Cm carboxylic acids” as used herein denotes in each case a linear or branched carboxylic acids having from n to m carbon atoms, such as 6 to 24 carbon atoms.

The term “Cn-Cm alcohols” as used herein denotes in each case a linear or branched alcohol having from n to m carbon atoms, such as having from 3 to 24 carbon atoms, or from 6 to 24 carbon atoms, or from 1 to 22 carbon atoms.

The term “C2-C12 dicarboxylic acids” as used herein denotes in each case a dicarboxylic acid having from 2 to 12 carbon atoms such as butanedioic acid (succinic acid), pentanedioic acid (glutaric acid), hexanedioic acid (adipic acid), or decanedioic acid (sebacic acid).

The term “dialkyl ether” as used herein denotes in each case a linear or branched dialkyl ether having a total of from 12 to 36 carbon atoms and comprising at least one ether moiety.

The term “C6-C22 alcohol carbonates” as used herein denotes in each case a linear or branched alcohol carbonates having 6 to 22 carbon atoms and comprising at least one functional group consisting of a carbonyl group flanked by two alkoxy groups.

The term “alkyl” as used herein denotes in each case a straight-chain or branched alkyl group having exemplarily from 1 to 18 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “alkoxy” as used herein denotes in each case a linear or branched alkyl group which is bonded via an oxygen atom and has usually from 1 to 20 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “carboxyalkyl” as used herein includes carboxymethyl, carboxyethyl, carboxypropyl, carboxyisopropyl, carboxybutyl, carboxyisobutyl, carboxyamyl, carboxyhexyl, carboxyheptyl, carboxyoctyl, carboxyisooctyl, carboxynonyl, carboxydecyl, carboxyundecyl, carboxydodecyl, carboxytetradecyl, carboxyhexadecyl, and carboxyoctadecyl, carboxymethyl being preferred.

The term “cycloalkyl” as used herein denotes in each case a monocyclic cycloaliphatic radical having usually from 3 to 10 or from 5 to 8 carbon atoms, such as cyclopropyl, cyclobutyl, cyclopentyl, cyclohexyl, cycloheptyl, cyclooctyl, cyclononyl and cyclodecyl or cyclopropyl, cyclobutyl, cyclopentyl and cyclohexyl.

The term “substituted” as used herein, means that a hydrogen atom bonded to a designated atom is replaced with a specified substituent, provided that the substitution results in a stable or chemically feasible compound. Unless otherwise indicated, a substituted atom may have one or more substituents and each substituent is independently selected.

The term “emollient” relates to cosmetic specific oils used for protecting, moisturizing and lubricating the skin. The word emollient is derived from the Latin word mollire, to soften. In general, emollients prevent evaporation of water from the skin by forming an occlusive coating. They can be divided into different groups depending on their polarity index.

The term “sensitive skin” refers to skin of which the natural barrier function is weakened and has broken due to a trigger. A trigger can be for example cold weather, extremely hot water and critical ingredients, which may be included in sunscreen or daily care compositions.

The term “sun protection factor (SPF)” as used herein indicates how well the skin is protected by a sunscreen composition mainly from UV-B radiation. In particular, the factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a thick dosage of 2 milligrams per square centimeter (mg/cm²).

The definition of “broadband” protection (also referred to as broad-spectrum or broad protection) is based on the “critical wavelength”. For broadband coverage, UV-B and UV-A protection must be provided. According to the US requirements, a critical wavelength of at least 370 nm is required for achieving broad spectrum protection. Furthermore, it is recommended by the European Commission that all sunscreen or cosmetic compositions should have an UV-A protection factor, which is at least one third of the labelled sun protection factor (SPF), e.g. if the sunscreen composition has an SPF of 30 the UVA protection factor has to be at least 10.

The term “critical wavelength” is defined as the wavelength at which the area under the UV protection curve (% protection versus wavelength) represents 90% of the total area under the curve in the UV region (290-400 nm). For example, a critical wavelength of 370 nm indicates that the protection of the sunscreen composition is not limited to the wavelengths of UV-B, i.e. wavelengths from 290-320 nm, but extends to 370 nm in such a way that 90% of the total area under the protective curve in the UV region are reached at 370 nm.

The term “administration” refers to the application of a sunscreen or daily care composition to the skin of a person.

As used herein the term “nano material” follows the recommendation of the European Commission 2011/696/EU. Accordingly, in a nano material 50% or more of particles, based on a number-based size distribution, are smaller than 100 nm, including constituent particles in aggregates or agglomerates. As such a threshold value of 50% is difficult to implement in regular production, in the spirit of this invention a non-nano UV absorber dispersion contains preferably less than 30% and more preferably less than 10% of particles smaller than 100 nm, referring to a number-based particle size distribution.

The term “dispersion” as used herein refers to a system in which distributed particles of one material are dispersed in a continuous phase of another material. The two phases may be in the same or different states of matter. A specific subtype of a dispersion is the “suspension”, wherein solid parts are dispersed (i.e. not dissolved) in a fluid.

Preferred embodiment regarding the process of manufacturing of an aqueous suspension (1) and of an aqueous suspension (1) as well as the use thereof are described hereinafter. It is to be understood that the preferred embodiments of the invention are preferred alone or in combination with each other.

As indicated above, the present invention relates in one embodiment to a process of manufacturing an aqueous suspension (1) of at least one organic UV filter having a particle size D_{N}30 of 100 nm or more, said process comprising the step of milling a suspension (2) comprising the at least one organic UV filter in a mixture of water and a hydrophobic additive in a milling apparatus at a temperature of 35 to 90° C.

The particle size D_{N}30 may be determined by e.g. transmission electron microscopy (TEM) or scanning electron microscopy (SEM), preferably by transmission electron microscopy (TEM). It is to be understood that an organic UV filter having a particle size D_{N}30 of 100 nm or more may also be expressed as an organic UV filter, wherein less than 30% of particles (number evaluation) are below 100 nm.

In a preferred embodiment A1 of the first aspect, the at least one organic UV filter in the aqueous suspension (1) has a particle size D_{N}10, preferably determined by transmission electron microscopy, of 100 nm or more, preferably of 0.1 to 0.4 μm, more preferably of 0.1 to 0.2 μm; and/or
a particle size D_{N}50, preferably determined by transmission electron microscopy, of 120 nm or more, preferably of from 0.12 to 0.5 μm, more preferably from 0.12 to 0.3 μm; and/or
a particle size D_{N}90, preferably determined by transmission electron microscopy, of 200 nm or more, preferably of from 0.2 to 1.0 μm, more preferably from 0.2 to 0.5 μm; and/or
a particle size Dv10 determined by laser diffraction of more than 0.1 μm, preferably of from 0.1 to 0.4 μm; and/or
a particle size Dv90 determined by laser diffraction of less than 2.2 μm, preferably of less than 2.0 μm, more preferably of less than 1.5 μm,
where Dv10 and Dv90 are measured using a Mastersizer 2000 from Malvern Panalytical.

In a preferred embodiment of the present invention, the at least one organic UV filter in the aqueous suspension (1) has a particle size Dv10 determined by laser diffraction using a Mastersizer 2000 from Malvern Panalytical of more than 0.1 μm to 0.4 μm, preferably of 0.15 to 0.3 μm; and/or
a particle size Dv50 determined by laser diffraction of from 0.2 μm to 0.8 μm, preferably of 0.4 to 0.6 μm; and/or
a particle size Dv90 determined by laser diffraction of from 0.5 μm to 3 μm, preferably of 0.5 to 2.2 μm.

In a preferred embodiment of the present invention, less than 10% of particles (number evaluation) are below 100 nm. Preferably the number evaluation is determined by transmission electron microscopy.

In a preferred embodiment of the present invention, the population of particles below 100 nm in the aqueous suspension (1) is reduced during the milling step. Preferably, the population of particles below 100 nm in the aqueous suspension (1) in the aqueous suspension (1) is reduced by increasing the temperature of the suspension (2) in the milling apparatus. The terms “is reduced” and “increasing the temperature” both relate to the comparison with a milling step that yields an aqueous suspension (1) with a D_{N}30 of 99 nm or less.

In a preferred embodiment of the present invention, the temperature of the suspension (2) in the milling step is in the range of 40 to 80° C., preferably of 40 to 70° C., and in particular of 45 to 65° C.

In a preferred embodiment of the present invention, the milling step is conducted for 1 to 40 hours, more preferred from 2 to 17 hours, or for 5 to 15 hours, or for 6 to 12 hours.

In a preferred embodiment of the present invention, the hydrophobic additive is a cosmetic oil, preferably selected from the group consisting of alcohols having from 6 to 18 carbon atoms; C6-C24 carboxylic acids; esters of C6-C24 carboxylic acids with C3-C24 alcohols; esters of hydroxycarboxylic acids with C6-C24 alcohols; esters of carboxylic acids with polyhydric alcohols; liquid mono-/di-/tri-glyceride mixtures based on C6-C18 carboxylic acids; esters of C6-C24 alcohols with aromatic carboxylic acids or with oxo carboxylic acids; tricarboxylic acid esters; esters of C2-C12 dicarboxylic acids with alcohols having from 1 to 22 carbon atoms or polyols having from 2 to 10 carbon atoms and from 2 to 6 hydroxy groups; substituted cyclohexanes; C6-C22 alcohol carbonates; symmetric or asymmetric dialkyl ethers having a total of from 12 to 36 carbon atoms; ring-opening products of epoxidized carboxylic acid esters with polyols; silicone oils; aliphatic or naphthenic hydrocarbons; diol esters; and mixtures thereof, in particular selected from the group consisting of esters of C6-C24 alcohols, preferably C10 to C17 alcohols, with aromatic carboxylic acids, preferably benzoic acid, or with hydroxycarboxylic acids, preferably lactate, or with oxo carboxylic acids, preferably levulinate; dicarboxylic acid esters, preferably di-n-butyl adipate; tricarboxylic acid esters, preferably tributyl citrate; and mixtures thereof.

Preferred C3-C24 alcohols are exemplarily isopropanol, n-butanol, iso-butanol, tert-amyl alcohol, n-hexanol, 3-methyl-3-pentanol, n-heptanol, n-octanol, n-nonanol, 2-ethylhexanol, decyl alcohol (capric alcohol), n-undecanol, lauryl alcohol, myristyl alcohol, cetyl alcohol, stearyl alcohol, oleyl alcohol, and behenyl alcohol.

Preferred C6-C24 alcohols are exemplarily n-hexanol, 3-methyl-3-pentanol, n-heptanol, n-octanol, n-nonanol, 2-ethylhexanol, decyl alcohol (capric alcohol), n-undecanol, lauryl alcohol, myristyl alcohol, cetyl alcohol, stearyl alcohol, oleyl alcohol, and behenyl alcohol.

Preferred are esters of C6-C24 alcohols, more preferably of C10-C20 alcohols, even more preferably C12 to C15 alcohols, with aromatic carboxylic acids, especially benzoic acid. Finsolv®TN may be named as a suitable ester of benzoic acid with linear and/or branched C6-C22 alcohols.

Preferred are monoesters of carboxylic acids with alcohols having from 3 to 24 carbon atoms. That group of substances comprises the esterification products of carboxylic acids having from 6 to 24 carbon atoms, for example caproic acid, caprylic acid, 2-ethylhexanoic acid, capric acid, lauric acid, isotride-canoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerisation of unsaturated fatty acids) with alcohols, for example isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl al-cohol, elaeostearyl alcohol, arachidyl alcohol, gadoleyl alcohol, behenyl alcohol, erucyl alcohol and brassidyl alcohol and technical-grade mixtures thereof (obtained, for example, in the high-pressure hydrogenation of technical-grade methyl esters based on fats and oils or aldehydes from Roelen's oxosynthesis and as monomer fractions in the dimerisation of unsaturated fatty alcohols). Of special importance are isopropyl myristate, isononanoic acid C16-C18 alkyl esters, stearic acid 2-ethylhexyl ester, cetyl oleate, glycerol tricaprylate, coconut fatty alcohol caprinate/caprylate and n-butyl stearate.

As a suitable ester of hydroxycarboxylic acids with C6-C24 alcohols dioctyl malates may be named.

As suitable polyhydric alcohols propylene glycol, dimer diol, or trimer triol may be named.

Preferably, triglycerides are based on C6-C10 carboxylic acids.

Preferred are esters of C2-C10 dicarboxylic acids, more preferably of C4-C8 dicarboxylic acids, with linear or branched alcohols having from 1 to 16 carbon atoms, more preferably having from 2 to 8 carbon atoms. Preferred dicarboxylic acid esters are di-n-butyl adipate, di(2-ethylhexyl) adipate, di(2-ethylhexyl) succinate and diisotridecyl acetate.

Preferred are linear or branched, symmetric or asymmetric dialkyl ethers having a total of from 12 to 24 carbon atoms, for example di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether, n-hexyl n-undecyl ether, di-tert-butyl ether, diisopentyl ether, di-3-ethyldecyl ether, tert-butyl n-octyl ether, isopentyl n-octyl ether and 2-methyl pentyl-n-octyl ether.

A preferred ester of C6-C24 alcohols with hydroxycarboxylic acids is lauryl lactate and a preferred ester of C6-C24 alcohols with oxo carboxylic acids is lauryl levulinate.

Preferred diol esters are ethylene glycol dioleate, ethylene glycol diisotridecanoate, propylene glycol di(2-ethylhexanoate), propylene glycol diisostearate, propylene glycol dipelargonate, butanediol diisostearate, and neopentyl glycol dicaprylate.

Preferred polyols are propylene glycol, hexylene glycol, glycerol and sorbitol.

In a preferred embodiment of the present invention, the hydrophobic additive is selected from alkyl benzoate such as C12 to C15 alkyl benzoate (e.g. commercially available as Cetiol AB), dibutyl adipat (e.g. Cetiol B), and mixtures thereof.

In a preferred embodiment of the present invention, the at least one organic UV filter is an insoluble organic UV filter. In this connection it is to be understood that the term insoluble UV filter refers to UV filters that are not soluble in water and cosmetic oils at 25° C. To the contrary, water soluble UV filters have a solubility in water of at least 2% by weight, preferably at least 3% by weight, more preferably at least 5% by weight and oil soluble UV filters have a solubility in common cosmetic oils, such as C₁₂-C₁₅-alkyl benzoate, dibutyl adipate, diisopropyl sebacate, phenethyl benzoate, or dicaprylyl carbonate of at least 2% by weight, preferably at least 5% by weight, more preferably at least 7% by weight.

In a preferred embodiment of the present invention, the at least one organic UV filter is selected from the group consisting of oxanilide UV filter, triazine UV filter, piperazine UV filter, triazole UV filter, vinyl group-containing amide UV filter, cinnamic acid amide UV filter, sulfonated benzimidazole, and mixtures thereof.

In a preferred embodiment of the present invention, the at least one organic UV filter is selected from the group consisting of oxanilide UV filter having the formula (1)

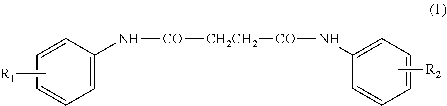

in which R₁ and R₂ are independently C1-C18 alkyl or C1-C18 alkoxy; triazine UV filter having the formula (2)

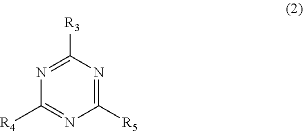

in which R₃, R₄ and R₅, independently, are H; OH; C1-C18 alkoxy; NH2; NH—R6 or N(R6)2 in which R6 is C1-C18 alkyl; OR6 in which R6 has its previous significance; phenyl; phenoxy; anilino; pyrrolo, in which the respective phenyl, phenoxy, anilino, or pyrrolo moieties are optionally substituted by one, two or three substituents selected from OH, carboxy, CO—NH2, C1-C18 alkyl or -alkoxy, C1-C18 carboxyalkyl, C5-C8 cycloalkyl, phenyl, a methylidenecamphor group, a group —(CH═CH)mC(═O)—OR6 in which m is 0 or 1 and R6 has its previous significance, or
a group

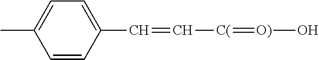

or the corresponding alkali metal, ammonium, mono-, di- or tri-C1-C4 alkylammonium, mono-, di- or tri-C2-C4 alkanolammonium salts, or the C1-C18alkyl esters thereof; phenylene bis-diphenyltriazine; a piperazine UV filter; a triazole UV filter having the formulae (31), (32) or (33)

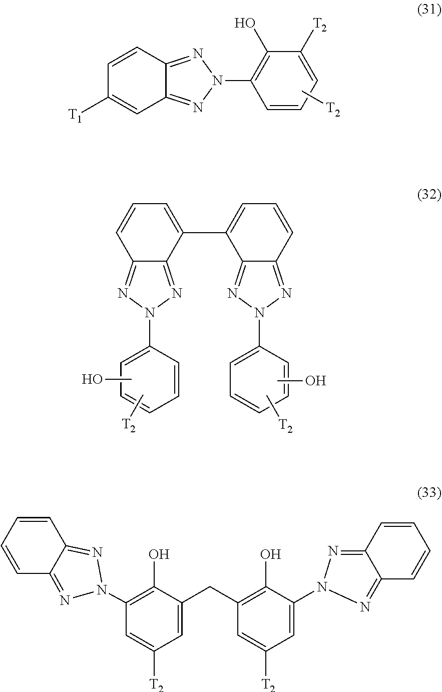

in which T₁ is C1-C18 alkyl or hydrogen and T₂ is C1-C18-alkyl, which is optionally substituted by phenyl; a vinyl group-containing amide UV filter having the formula (4)

R₉—(Y)ₘ—CO—C(R₁₀)═C(R₁₁)—N(R₁₂)(R₁₃) (4)

in which R₉ is C1-C18 alkyl or phenyl optionally substituted by one, two or three substituents selected from OH, C1-C18 alkyl, C1-C18 alkoxy or CO—OR6 in which R6 has its previous significance; R₁₀, R₁₁, R₁₂ and R₁₃ are the same or different and each is independently C1-C18 alkyl or hydrogen; Y is N or O; and m has its previous significance; cinnamic acid amide UV filter having the formula (5)

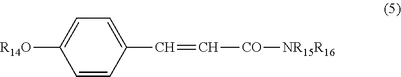

in which R₁₄ is hydroxy or C1-C4 alkoxy; R₁₅ is hydrogen or C1-C4 alkyl; and R₁₆ is —(CONH)m-phenyl in which m has its previous significance and the phenyl group is optionally substituted by one, two or three substituents selected from OH, C1-C18 alkyl, C1-C18 alkoxy or CO—OR6 in which R6 has its previous significance; sulfonated benzimidazole UV filter having the formula (6)

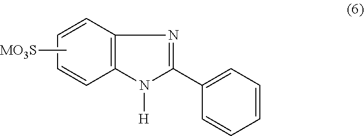

in which M is hydrogen or an alkali metal, an alkaline earth metal or zinc; and mixtures thereof.

In a preferred embodiment of the present invention, the at least one organic UV filter is selected from the group consisting of oxanilide UV filter having the formula (1)

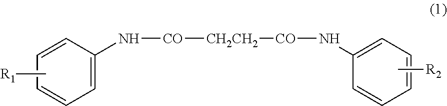

in which R₁ and R₂ are independently C1-C18 alkyl or C1-C18 alkoxy; triazine UV filter having the formula (2)

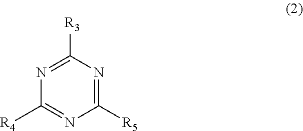

in which R₃, R₄ and R₅, independently, are H; OH; C1-C18 alkoxy; NH2; NH—R6 or N(R6)2 in which R6 is C1-C18 alkyl; OR6 in which R6 has its previous significance; phenyl; phenoxy; anilino; pyrrolo, in which the respective phenyl, phenoxy, anilino, or pyrrolo moieties are optionally substituted by one, two or three substituents selected from OH, carboxy, CO—NH2, C1-C18 alkyl or -alkoxy, C1-C18 carboxyalkyl, C5-C8 cycloalkyl, phenyl, a methylidenecamphor group, a group —(CH═CH)mC(═O)—OR6 in which m is 0 or 1 and R6 has its previous significance, or
a group

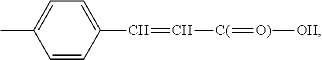

or the C1-C18alkyl esters thereof; phenylene a group bis-diphenyltriazine; a piperazine UV filter; a triazole UV filter having the formulae (31), (32) or (33)

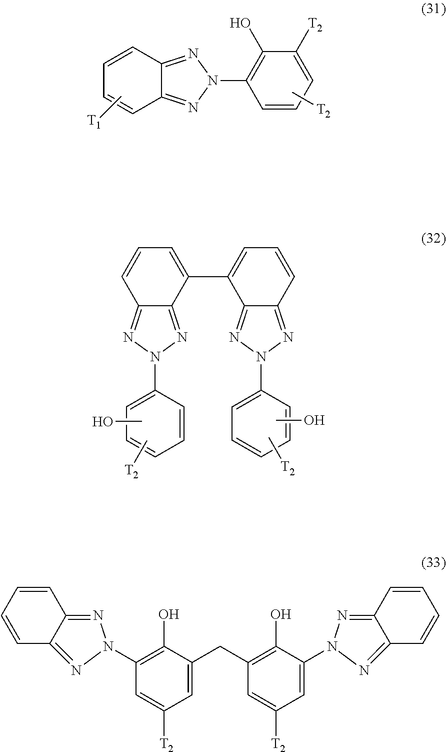

in which T₁ is C1-C18 alkyl or hydrogen and T₂ is C1-C18-alkyl, which is optionally substituted by phenyl; a vinyl group-containing amide UV filter having the formula (4)

R₉—(Y)ₘ—CO—C(R₁₀)═C(R₁₁)—N(R₁₂)(R₁₃) (4)

in which R₉ is C1-C18 alkyl or phenyl optionally substituted by one, two or three substituents selected from OH, C1-C18 alkyl, C1-C18 alkoxy or CO—OR6 in which R6 has its previous significance; R₁₀, R₁₁, R₁₂ and R₁₃ are the same or different and each is independently C1-C18 alkyl or hydrogen; Y is N or O; and m has its previous significance; cinnamic acid amide UV filter having the formula (5)

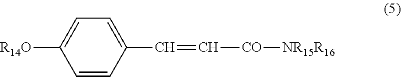

in which R₁₄ is hydroxy or C1-C4 alkoxy; R₁₅ is hydrogen or C1-C4 alkyl; and R₁₆ is —(CONH)m-phenyl in which m has its previous significance and the phenyl group is optionally substituted by one, two or three substituents selected from OH, C1-C18 alkyl, C1-C18 alkoxy or CO—OR6 in which R6 has its previous significance; sulfonated benzimidazole UV filter having the formula (6)

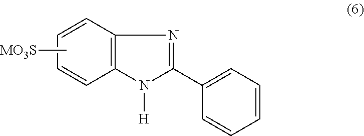

in which M is hydrogen; and mixtures thereof.

In a preferred embodiment of the present invention, the at least one organic UV filter is selected from the group consisting of tris-biphenyl triazine, 1,1′-(1,4-piperazinediyl)bis[1-[2-[4-(diethylamino)-2-hydroxybenzoyl]phenyl]-methanone, phenylene bis-diphenyltriazine, 2,2′-methylenebis[6-(2H-1,2,3-benzotriazol-2-yl)-4-(2,4,4-trimethylpentan-2-yl)phenol], and mixtures thereof, and in particular wherein the at least one organic UV filter is selected from the group consisting of tris-biphenyl triazine and 2,2′-methylenebis[6-(2H-1,2,3-benzotriazol-2-yl)-4-(2,4,4-trimethylpentan-2-yl)phenol].

In a preferred embodiment of the present invention, the at least one organic UV filter is 2,2′-methylenebis [6-(2H-1,2,3-benzotriazol-2-yl)-4-(2,4,4-trimethylpentan-2-yl)phenol].

In a preferred embodiment of the present invention, the at least one organic UV filter is a broadband filter.

In a preferred embodiment of the present invention, the hydrophobic additive is comprised in the aqueous suspension (1) in an amount of 0.01 to 10.0 wt.-%, preferably of 0.01 to 5.0 wt.-%, more preferably of 0.01 to 3.0 wt.-%, and in particular of 0.05 to 1.0 wt.-%, based on the total amount of the aqueous suspension (1).

In another preferred embodiment of the present invention, the hydrophobic additive is comprised in the aqueous suspension (1) in an amount of 0.001 to 10.0 wt.-%, preferably of 0.05 to 5.0 wt.-%, more preferably of 0.1 to 3.0 wt.-%, and in particular of 0.2 to 1.0 wt.-%, based on the total amount of the aqueous suspension (1).

In a preferred embodiment of the present invention, the aqueous suspension (1) is free of an alkyl polyglucoside.

In a preferred embodiment of the present invention, the aqueous suspension (1) further comprises a dispersing agent.

Suitable dispersing agents are polyglycerol alkyl ester, preferably polyglycerol monoalkyl ester, and alkyl polyglucoside, preferably having the formula CₙH₂ₙ₊₁O(C₆H₁₀O₅)ₓH, in which n is an integer ranging from 8 to 16 and x is the mean polymerization level of the glucoside moiety (C₆H₁₀O) and ranges from 1.4 to 1.6, or an ester thereof. Further suitable dispersing agents are disclosed in WO2009068469.

According to the present invention, the polyglycerol monoalkyl ester has preferably a mean degree of polymerization of glycerol of 5 or more.

In one preferred embodiment, the at least one polyglycerol monoalkyl ester is selected from the group consisting of decaglyceryl caprate, decaglyceryl monolaurate, decaglyceryl myristate, decaglyceryl oleate, decaglyceryl stearate, decaglyceryl isostearate, hexaglyceryl cap rate, hexaglyceryl laurate, hexaglyceryl myristate, hexaglyceryl oleate, hexaglyceryl stearate, hexaglyceryl isostearate, pentaglyceryl caprate, pentaglyceryl laurate, pentaglyceryl myristate, pentaglyceryl oleate, pentaglyceryl stearate, pentaglyceryl isostearate, and combinations thereof. In a particular preferred embodiment, the at least one polyglycerol monoalkyl ester is decaglyceryl monolaurate (INCI polyglyceryl-10 laurate).

Polyglycerol monoalkyl esters having an HLB (hydrophilic-lipophilic balance) of 14.5 or more are preferable and having an HLB of 15 or more are more preferable. The HLP value is determined by the formula

$$HLB = 20 \cdot {M}_{h} / M ,$$

wherein Mₙ is the molecular mass of the hydrophilic portion of the molecule and M is the molecular mass of the whole molecule.

Polyglycerol monoalkyl esters having an HLB of less than 14.5 may take a longer time for dispersion of micronized methylene bis-benzotriazolyl tetramethylbutylphenol in water phase components. Examples of polyglycerol monoalkyl esters with a mean degree of polymerization of 5 or more and having an HLB of 14.5 or more may include decaglyceryl caprate, decaglyceryl monolaurate, decaglyceryl myristate, decaglyceryl oleate, decaglyceryl stearate, decaglyceryl isostearate, hexaglyceryl laurate, pentaglyceryl laurate, pentaglyceryl myristate, pentaglyceryl stearate, and pentaglyceryl oleate, and those having an HLB of 15 or more may include decaglyceryl caprate and decaglyceryl monolaurate.

Particularly preferred are polyglyceryl monolaurate, particularly decaglyceryl monolaurate, and decyl glucoside.

Preferably, the alkyl polyglucoside consists of a C1-C12ester of the compound of formula CₙH₂ₙ₊₁O(C₆H₁₀O₅)ₓH, namely an ester formed by reacting a C1-C12 carboxylic acid with one or more free PH group of the glucoside moiety (C₆H₁₀O). In this connection, it is preferred that the ester is formed by reacting formic, acetic, propionic, butyric, sulfosuccinic, citric, or tartaric acid, with one or more free OH groups on the glucoside moiety (C₆H₁₀O).

Preferably, the aqueous suspension (1) comprises the dispersing agent in an amount of 1 to 15 wt.-%, more preferably of 5 to 40 wt.-%, and in particular of 6 to 20 wt.-%, based on the total weight of the aqueous suspension (1).

The weight ratio of the dispersing agent to the at least one organic UV filter is preferably from 0.05 to 0.5, more preferably from 0.08 to 0.4, and in particular from 0.1 to 0.3.

Further excipients may be added to the preparation, such as thickeners, anti-foaming agents, pH-adjusters, preservatives.

Examples of further excipients which may be used for the preparation of the micronized at least one organic UV filter are rheology modifiers, solvents, pH adjuster or buffering agents, antifoaming agents, and preservatives.

Rheology modifiers are optionally added to the UV protection composition which help to stabilize across the time such composition. Examples for aqueous thickeners are represented by natural ingredients and their derivatives such as gums and alginates or by synthetic/semi-synthetic ingredients such as modified starch, modified cellulose and polyacrylates. A preferred rheology modifier is xanthan gum.

Suitable solvents for the grinding process are water, brine, (poly-)ethylenglycol, glycerine or cosmetically acceptable oils. Other suitable solvents are disclosed in the IPCOM N°000031257D in the chapters “esters of fatty acids”, “natural and synthetic triglycerides including glyceryl esters and derivatives”, “perlescent waxes”, “hydrocarbon oils”, and “silicones or siloxanes”. Preferred solvents are water, butylene glycol, and caprylyl glycol.

Suitable pH adjusters are NaOH, TEA, and citric acid.

A suitable antifoaming agent is simethicone.

A suitable preservative is COSING Annex V.

In a preferred embodiment of the present invention, the milling step is conducted in a ball mill, a vibratory mill, a wet rotor mill, a stirred media mill, or a colloid mill, preferably a wet rotor mill or a stirred media mill, and in particular in a stirred media mill with milling beads, preferably glass beads, zirconium oxide, or mixed ceramic grinding beads, having a diameter of 0.1 to 10 mm, preferably of 0.15 to 5 mm, and in particular of 0.2 to 3 mm.

Preferably, the grinding beads are yttrium-stabilized zirconium oxide, more preferably having a high density and are highly spherical.

Typical yttrium-stabilized zirconium oxide grinding beads according to the present invention have the following properties:
Chemical Composition: 95% ZrO2, 5% Y2O3
Specific Density: 6.1g/cm³
Bending Strength: 1200 MPa
Hardness (Hv10): 1250
Modulus of Elasticity: 210 GPa
Fracture Toughness: 6.0 Mpam°

Such grinding beads are e.g. commercially available at Tosho Ceramics, Japan.

The process according to the present invention may also be referred to as micronization process.

Usually, the micronization process starts with the solid UV filter raw material which is obtained from synthesis and optionally the respective workup process. Typical process steps involve for example crystallization or re-crystallization steps to remove impurities or to achieve a particular crystal structure, solid/liquid separation steps such as filtration, purification steps such as washing of the UV filter particles, drying steps to remove residual solvents. Processing steps such as agglomeration or re-agglomeration to improve flow behavior, increase shelf life of the material, or reduce dust formation in handling may also be considered. The UV filter material may be subjected to pre-grinding to break lumps or agglomerates of crystals or even break crystals to smaller size. Preferably, the UV filter material will be available for micronization as a powder or a granule, but also other solid forms such as agglomerated crystals can be processed.

The UV filter material is then formulated into a liquid slurry suitable for wet grinding. The slurry contains the solvent, preferably water, and compounds that are capable of wetting the at least one organic UV filter solids in the solvent. The slurry is prepared in equipment which allows the preparation of the liquid formulation, the incorporation of the at least one organic UV filter and the formation of a homogenous dispersion for further processing. For wet grinding, the slurry contains dispersing agents, such as surfactants, emulsifiers, and/or polymers, enabling to stabilize the crystal surface. The dispersing agents may be formulated at once into the slurry or may be dosed into the slurry during the process. The slurry may contain further excipients such as organic solvents, preservatives, pH adjusters or buffering agents, anti-foaming agents and so on.

For executing the invention, it is necessary that the hydrophobic additive is present when the dispersion passes through the milling device.

Micronization of the at least one organic UV filter dispersion may be conducted in more than one processing step. Depending on the properties of the at least one organic UV filter and the target particle size of the dispersion, the selected equipment may require specific properties of the slurry. For example, to operate a stirred ball mill a maximum particle size in the dispersion must not be exceeded to prevent mill blocking. For this reason, one may use a pre-milling device such as a colloid mill, or use more than one stirred ball mill along the processing line. The expert skilled in the art will be able to design equipment and process in such a way that an economic and efficient production is achieved.

In a preferred embodiment of the present invention, the at least one organic UV filter in suspension (2) has a particle size Dv90 determined by laser diffraction in the range of from 0.01 to 300 μm, preferably from 0.1 to 250 μm.

In principle, all devices used for wet grinding can be used for executing the invention. Preferably, the particle size of the inventive aqueous suspension (1) comprising at least one organic UV filter is adjusted in such a way that product performance is high while the non-nano attribute is safely met in production. Shear/stress forces which are generated in stirred ball mills are capable to break particles down to nano size. However, the invention can also be used to suppress the build-up of nanoparticles that may be generated when the at least one organic UV filter particle is subjected to impact forces, for example in wet rotor mills.

Most preferable, the invention is carried out in a device containing grinding beads, such as a stirred ball mill.

In a preferred embodiment of the present invention, a trigger mechanism is needed to activate the effect of the hydrophobic additive. Specifically, the inventive effect takes place only at elevated product temperature which provides an effective switch on/off mechanism by adjusting the product temperature in the grinding apparatus.

Without being bound by theory, it is speculated that the joint action of hydrophobic additive and temperature enhances Ostwald-ripening of the UV absorber particles. This leads to dissolution of nano particles and induces crystal growth which counteracts the grinding action in the mill. Ostwald ripening may then be effectively suppressed by cooling the dispersion after it leaves the mill. The expert skilled in the art will find combinations of process parameters to achieve the non-nano particle size distribution in target.

As mentioned above, the invention further relates in a second aspect to an aqueous suspension (1) comprising
a) 10 to 65 wt.-% of at least one organic UV filter having a particle size D_{N}30 of 100 nm or more
b) 0.01 to 10.0 wt.-% of a hydrophobic additive selected from the group consisting of alcohols having from 6 to 18 carbon atoms; C6-C24 carboxylic acids; esters of C6-C24 carboxylic acids with C3-C24 alcohols; esters of hydroxycarboxylic acids with C6-C24 alcohols; esters of carboxylic acids with polyhydric alcohols; liquid mono-/di-/tri-glyceride mixtures based on C6-C18 carboxylic acids; esters of C6-C24 alcohols with aromatic carboxylic acids or with oxo carboxylic acids; tricarboxylic acid esters; esters of C2-C12 dicarboxylic acids with alcohols having from 1 to 22 carbon atoms or polyols having from 2 to 10 carbon atoms and from 2 to 6 hydroxy groups; substituted cyclohexanes; C6-C22 alcohol carbonates; symmetric or asymmetric dialkyl ethers having a total of from 12 to 36 carbon atoms; ring-opening products of epoxidized carboxylic acid esters with polyols; silicone oils; aliphatic or naphthenic hydrocarbons; diol esters; and mixtures thereof, and
c) water
   each based on the total amount of the aqueous suspension (1).

In a preferred embodiment of the present invention, the hydrophobic additive is selected from the group consisting of esters of C6-C24 alcohols, preferably C10 to C17 alcohols, with aromatic carboxylic acids, preferably benzoic acid, or with hydroxycarboxylic acids, preferably lactate, or with oxo carboxylic acids, preferably levulinate; dicarboxylic acid esters, preferably di-n-butyl adipate; tricarboxylic acid esters, preferably tributyl citrate; and mixtures thereof.

In a preferred embodiment of the present invention, the at least one organic UV filter is an insoluble organic UV filter, preferably selected from the group consisting of oxanilide UV filter, triazine UV filter, piperazine UV filter, triazole UV filter, vinyl group-containing amide UV filter, cinnamic acid amide UV filter, sulfonated benzimidazole, and mixtures thereof, more preferably selected from the group consisting of oxanilide UV filter having the formula (1)

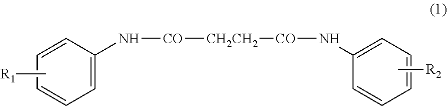

in which R₁ and R₂ are independently C1-C18 alkyl or C1-C18 alkoxy; triazine UV filter having the formula (2)

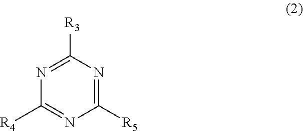

in which R₃, R₄ and R₅, independently, are H; OH; C1-C18 alkoxy; NH2; NH—R6 or N(R6)2 in which R6 is C1-C18 alkyl; OR6 in which R6 has its previous significance; phenyl; phenoxy; anilino; pyrrolo, in which the respective phenyl, phenoxy, anilino, or pyrrolo moieties are optionally substituted by one, two or three substituents selected from OH, carboxy, CO—NH2, C1-C18 alkyl or -alkoxy, C1-C18 carboxyalkyl, C5-C8 cycloalkyl, phenyl, a methylidenecamphor group, a group —(CH═CH)mC(═O)—OR6 in which m is 0 or 1 and R6 has its previous significance, or
a group

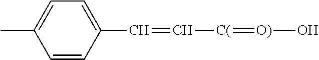

or the corresponding alkali metal, ammonium, a group mono-, di- or tri-C1-C4 alkylammonium, mono-, di- or tri-C2-C4 alkanolammonium salts, or the C1-C18alkyl esters thereof; phenylene bis-diphenyltriazine; a piperazine UV filter; a triazole UV filter having the formulae (31), (32) or (33)

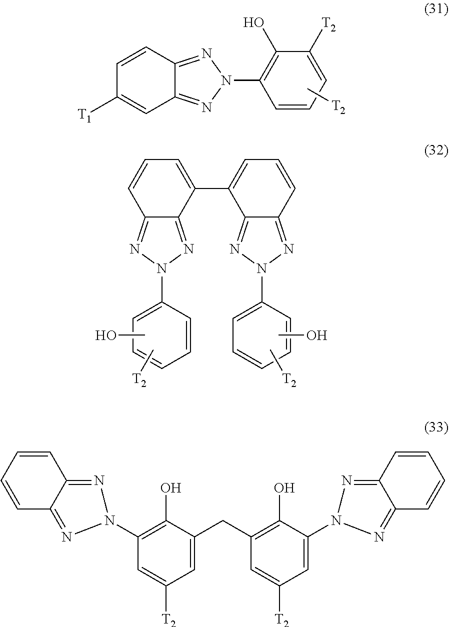

in which T₁ is C1-C18 alkyl or hydrogen and T₂ is C1-C18-alkyl, which is optionally substituted by phenyl; a vinyl group-containing amide UV filter having the formula (4)

R₉—(Y)ₘ—CO—C(R₁₀)═C(R₁₁)—N(R₁₂)(R₁₃) (4)

in which R₉ is C1-C18 alkyl or phenyl optionally substituted by one, two or three substituents selected from OH, C1-C18 alkyl, C1-C18 alkoxy or CO—OR6 in which R6 has its previous significance; R₁₀, R₁₁, R₁₂ and R₁₃ are the same or different and each is independently C1-C18 alkyl or hydrogen; Y is N or O; and m has its previous significance; cinnamic acid amide UV filter having the formula (5)

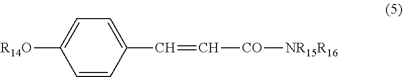

in which R₁₄ is hydroxy or C1-C4 alkoxy; R₁₅ is hydrogen or C1-C4 alkyl; and R₁₆ is —(CONH)m-phenyl in which m has its previous significance and the phenyl group is optionally substituted by one, two or three substituents selected from OH, C1-C18 alkyl, C1-C18 alkoxy or CO—OR6 in which R6 has its previous significance; sulfonated benzimidazole UV filter having the formula (6)

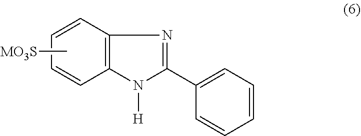

in which M is hydrogen or an alkali metal, an alkaline earth metal or zinc; and mixtures thereof, even more preferably wherein the at least one organic UV filter is selected from the group consisting of tris-biphenyl triazine, 1,1′-(1,4-piperazinediyl)bis[1-[2-[4-(diethylamino)-2-hydroxybenzoyl]phenyl]-methanone, phenylene bis-diphenyltriazine, and 2,2′-methylenebis [6-(2H-1,2,3-benzotriazol-2-yl)-4-(2,4,4-trimethylpentan-2-yl)phenol], and in particular wherein the at least one organic UV filter is selected from the group consisting of tris-biphenyl triazine and 2,2′-methylenebis [6-(2H-1,2,3-benzotriazol-2-yl)-4-(2,4,4-trimethylpentan-2-yl)phenol].

In a preferred embodiment of the present invention, the aqueous suspension (1) comprises the hydrophobic additive in an amount of 0.01 to 8.0 wt.-%, more preferably of 0.01 to 5.0 wt.-%, more preferably of 0.01 to 3.0 wt.-%, and in particular of 0.05 to 1.0 wt.-%, based on the total amount of the aqueous suspension (1).

In another preferred embodiment of the present invention, the hydrophobic additive is comprised in the aqueous suspension (1) in an amount of 0.05 to 5.0 wt.-%, preferably of 0.1 to 3.0 wt.-%, and in particular of 0.2 to 1.0 wt.-%, based on the total amount of the aqueous suspension (1).

In a preferred embodiment of the present invention, the at least one organic UV filter has a particle size D_{N}10, preferably determined by transmission electron microscopy, of 100 nm or more, preferably of 0.1 to 0.4 μm, more preferably of 0.1 to 0.2 μm; and/or
a particle size D_{N}50, preferably determined by transmission electron microscopy, of 120 nm or more, preferably of from 0.12 to 0.5 μm, more preferably from 0.12 to 0.3 μm; and/or
a particle size D_{N}90, preferably determined by transmission electron microscopy, of 200 nm or more, preferably of from 0.2 to 1.0 μm, more preferably from 0.2 to 0.5 μm; and/or
a particle size Dv10 determined by laser diffraction of more than 0.1 μm, preferably of from 0.1 to 0.4 μm; and/or
a particle size Dv90 determined by laser diffraction of less than 2.2 μm, preferably of less than 2.0 μm, more preferably of less than 1.5 μm,
where Dv10 and Dv90 are measured using a Mastersizer 2000 from Malvern Panalytical.

In a preferred embodiment of the present invention, the at least one organic UV filter has a particle size D_{N}10, preferably determined by transmission electron microscopy, of 100 nm or more, preferably of 0.1 to 0.4 μm, more preferably of 0.1 to 0.2 μm; and/or
a particle size D_{N}50, preferably determined by transmission electron microscopy, of 150 nm or more, preferably of from 0.15 to 0.5 μm, more preferably from 0.15 to 0.3 μm; and/or
a particle size D_{N}90, preferably determined by transmission electron microscopy, of 200 nm or more, preferably of from 0.2 to 1.0 μm, more preferably from 0.2 to 0.5 μm; and/or
a particle size Dv10 determined by laser diffraction of more than 0.1 μm; and/or
a particle size Dv90 determined by laser diffraction of less than 2.2 μm.

In a preferred embodiment of the present invention, the at least one organic UV filter has a specific surface area determined by laser diffraction of 8 to 32 m²/g, preferably of 10 to 30 m²/g, and in particular of 13 to 28 m²/g.

Further preferred embodiments of the hydrophobic additive, the at least one organic UV filter, the dispersing agent, the further excipients, and the like, and the amounts of the remaining components have already been described above and apply for the aqueous suspension (1), as well.

As mentioned above, the invention further relates in a third aspect to the herein described aqueous suspension (1) for use in sunscreen or daily care composition.

It is to be understood that the preferred embodiments of the aqueous suspension (1) such as the hydrophobic additive, the at least one organic UV filter, the dispersing agent, the further excipients, and the like, and the amounts of the components, have already been described above and apply for the use, as well.

The sunscreen or daily care compositions as disclosed herein are suitable for the administration on any skin type, in particular for the administration on sensitive skin.

### EXAMPLES

### Comparative Example A

Micronization of 2,2′-methylenebis [6-(2H-1,2,3-benzotriazol-2-yl)-4-(2,4,4-trimethylpentan-2-yl)phenol] (also known as methylene bis-benzotriazolyl tetramethylbutylphenol and MBBT) dispersion

600 g of Plantacare® 2000 UP (alkyl polyglucoside solution, wherein the alkyl polyglucoside is decyl glucoside, active material 50%, from BASF) were dissolved in 1400 g of deionized water. In this solution, 2000 g of Tinosorb MBBT (MBBT, from BASF) were added and homogenized. The dispersion was then milled at 26° C. in a stirred ball mill (Dyno Mill Multilab, from Bachofen) equipped with 1.4 L milling container and ceramic discs. 2 mm glass beads were used as the grinding beads, and the dispersion was circulated through the mill during the grinding process. The process was stopped after 90 minutes, total grinding energy was 0.93 kWh. The product was separated from the grinding beads.

Volume based particle size distribution was measured by laser diffraction (Mastersizer 2000, from Malvern Panalytical). The specific absorbance E(1,1) was measured with a Lambda 25 spectrometer equipped with Ulbricht Kugel. E(1,1) was related to the UV filter performance of the dispersion. Specific surface area (SSA) was determined by laser diffraction (Mastersizer 2000, from Malvern Panalytical).

### Results:

Particle size (volume-based): Dv10-Dv50-Dv90 (nm)=85-205-1583; SSA=33.9 m²/g E(1,1)=329

In the following examples, UV Absorber 1 denotes MBBT as in example 1, UV Absorber 2 denotes 1,3,5-Triazine, 2,4,6-tris [1,1′-biphenyl]-4-yl-, UV Absorber 3 denotes Bis-(Diethylaminohydroxybenzoyl Benzoyl) Piperazine and UV Absorber 4 denotes 3,3′-(1,4-Phenylene)bis(5,6-diphenyl-1,2,4-triazine).

### Comparative Examples B1-B10

### Wet Milling of UV Absorber Dispersions

UV absorbers were micronized in water using different formulations and milling equipment. Micronization progress was monitored by taking two samples a) and b) during the milling process. Volume-based particle size distribution was obtained from laser diffraction. The results are summarized in Table 1.

TABLE 1
Different milling conditions and results thereof; PSD results are volume-based.

| | | | |
|---|---|---|---|
| | | | Laser diffraction |
| | | Milling | Dᵥ10-Dᵥ50-Dᵥ90 (nm); SSA |
| No. | Composition (wt-%) | Equipment | (m²/g) |

UV Absorber 1

| | | | |
|---|---|---|---|
| B1 | 50% UV Absorber 1, 8% alkyl | Dyno Multilab; | a) 98-848-3210; 23.1 |
| | polyglucoside, water to 100 | Y—ZrO2 beads, | b) 83-211-2129; 32.9 |
| | | 1.4-1.6 mm | |
| B2 | 50% UV Absorber 1, 7.5% | Dyno Multilab; | a) 78-205-1943 |
| | alkyl polyglucoside, water to | Y—ZrO2 beads, | b) 70-150-1100 |
| | 100 | 0.6-0.8 mm | |
| B3 | 50% UV Absorber 1, 5% | Beaker mill; | a) 83-205-724; 36.8 |
| | Sodium cocoyl glutamate, | Glass beads, | b) 77-159-349; 43.5 |
| | 0.5% caprylyl glycol, 0.01% | 1 mm | |
| | defoamer, water to 100 | | |
| B4 | 50% UV Absorber 1, 8% | Dyno Multilab; | a) 91-972-4571; 24.6 |
| | decaglyceryl monolaurate, 7% | Y—ZrO2 beads, | b) 86-268-2975; 30.2 |
| | butylene glycole, water to 100 | 1.0-1.2 mm | |

UV Absorber 2

| | | | |
|---|---|---|---|
| B5 | 50% UV Absorber 2, 7.5% | Dyno Multilab; | a) 97-523-1978; 24.3 |
| | alkyl polyglucoside, water to | Y—ZrO2 beads, | b) 81-195-1142; 37.8 |
| | 100 | 0.3-0.4 mm | |
| B6 | 50% UV Absorber 2, 10% | Beaker mill; | a) 112-615-1584; 20.5 |
| | Lauryl alcohol alkoxylate, | Glass beads, | b) 111-249-541; 29.2 |
| | water to 100 | 0.5-0.8 mm | |

UV Absorber 3

| | | | |
|---|---|---|---|
| B7 | 50% UV Absorber 3, 6% alkyl | LMZ 10; | a) 62-140-724 |
| | polyglucoside, 1% sodium | Y—ZrO2 beads, | b) 58-91-168 |
| | lauryl myristyl ether sulfate, | 0.3-0.4 mm | |
| | 0.2% defoamer, water to 100 | | |
| B8 | 50% UV Absorber 3, 5% | Dyno Multilab; | a) 83-273-1834; 23.9 |
| | disodium C12-18 alkyl | Y—ZrO2 beads, | b) 71-138-905; 35.6 |
| | sulfosuccinate, 1% Sodium | 1.0-1.2 mm | |
| | Cocoyl Hydrolyzed Wheat | | |
| | Protein, water to 100 | | |
| B9 | 50% UV Absorber 3, 5% | Beaker mill; | a) 80-169-1241; 28.6 |
| | Sodium cocoyl glutamate, 1% | Y—ZrO2 beads, | b) 70-131-574; 37.8 |
| | Sodium Cocoyl Hydrolyzed | 1.0-1.2 mm | |
| | Wheat Protein, water to 100 | | |

UV Absorber 4

| | | | |
|---|---|---|---|
| B10 | 50% UV Absorber 4, 10% | Beaker mill; | a) 69-126-757; 38.7 |
| | Lauryl alcohol alkoxylate, | Y—ZrO2 beads, | b) 63-109-194; 46.7 |
| | water to 100 | 1.0-1.2 mm | |

Micronization of UV absorber dispersions works with different equipment and formulations. The progress in micronization is evident in the decrease of PSD results (especially Dv50, Dv90) and increase of SSA. Due to the limited sensitivity of laser diffraction for nanoparticles, the Dv10 value is only slightly decrease.

### Comparative Example C1

A micronized dispersions containing 50% of UV Absorber 1 and 7.5% of alkyl polyglucoside was analyzed using laser diffraction and TEM. Results are summarized in Table 2. The milling process was performed in a stirred media mill (LMZ) using Y—ZrO2 beads.

TABLE 2
Comparison of PSD (laser diffraction)
and PSD (TEM) values of Sample C1.

| | PSD (laser diffraction) | |
|---|---|---|
| | Dᵥ10-Dᵥ50-Dᵥ90 (nm); SSA | PSD (TEM) |
| Example | (m²/g) | number distribution |
| Material C1 | 99-326-2400; 26 | DN50: 115.5 nm |
| | | Particles < 100 nm: 37% |

TEM analysis yields a number distribution and is suitable for applying the nanomaterial criteria according to 2011/696/EU.

The result demonstrates that material C1 is borderline/close to the nano regime even though Dv10 from laser light diffraction is about 100 nm and Dv90 is well above 2 μm. Comparison with Table 1 suggests that most or all of the materials in example B1-B10 are nanomaterial.

### Comparative Examples C2-C4: Addition of Oils

Material C1 was taken as starting material.

Oil was added in examples C2 to C4 and the milling process was continued. A beaker mill with 2 mm glass beads was used for all experiments. Temperature of the product was controlled during the milling process. Samples were taken at the indicated duration of milling (eg. 4 hours) and PSD was measured.

TABLE 3
Addition of sun flower oil; PSD results are volume-based.

| | | | Laser diffraction |
|---|---|---|---|
| | wt-% of added | | Dᵥ10-Dᵥ50-Dᵥ90 (nm); SSA |
| No. | oil to C1 | T(° C.) | (m²/g) |
| C2 | no additives | 49 | 2 h 20: 93-257-1584; 29.9 |
| | | | 5 h 20: 94-240-1120; 31.4 |
| | | | 8 h: 90-225 863; 33.5 |
| C3 | 0.1% of sun | 49 | 4 h: 94-241-1136; 31.4 |
| | flower oil | | 8 h: 86-210-681; 35.9 |
| | | | 10 h: 82-197-513; 38.1 |
| | | 10-12 h: 22° C. | 12 h: 80-188-455; 39.7 |
| C4 | 0.5% of sun | 52 | 4 h: 93-236-1075; 32.0 |
| | flower oil | | 8 h: 85-207-582; 36.4 |
| | | | 10 h: 80-190-462; 39.4 |
| | | 10-12 h: 22° C. | 12 h: 79-183-422; 40.7 |

Addition of small amounts of sunflower oil has no significant impact on the micronization progress. PSD decreases with duration of milling, irrespective of the temperature of the product in the mill.

### Examples According to the Invention C5-C10

Material C1 was taken as starting material.

Oil was added in examples C5 to C10 and the milling process was continued. A beaker mill with 2 mm glass beads was used for all experiments. Temperature of the product was controlled during the milling process. Samples were taken at the indicated duration of milling (eg. 2 hours) and PSD was measured.

TABLE 4
Addition of Cetiol AB (C12-15 alkyl benzoate);
PSD results are volume-based.

| | | | Laser diffraction |
|---|---|---|---|
| | wt-% of added | | Dᵥ10- Dᵥ50- Dᵥ90 (nm); SSA |
| No. | oil to C1 | T(° C.) | (m²/g) |
| C5 | 0.1% Cetiol AB | 54 | 2 h: 102-280-1710; 27.4 |
| | | | 5 h 30: 96-245-1170; 30.8 |
| | | | 7 h: 95-241-1000; 31.5 |
| | | | 9 h 30: 134-298-1070; 23.8 |
| C6 | 0.25% Cetiol AB | 52 | 2 h: 105-293-1850; 26.3 |
| | | | 5 h 30: 103-266-1460; 28.3 |
| | | | 7 h: 102-259-1240; 29.0 |
| | | | 9 h 30: 141-308-1130; 22.6 |
| C7 | 0.5% Cetiol AB | 60 | 2 h: 107-311-2110; 25.3 |
| | | | 5 h 30: 204-492-2100; 14.3 |

In contrast to examples C2-C4, Dᵥ50 and Dᵥ90 decrease less within short milling durations. When the process is continued, Dᵥ10 and Dᵥ50 start to increase which is attributed to particle growth during micronization process.

The final material obtained in example C7 was further analyzed.

E(1,1) was measured with a Lambda 25 spectrometer equipped with Ulbricht Kugel.

TEM analysis was carried out to obtain number based particle size distribution and particle morphology. Results were compiled in Table 5.

TABLE 5
Comparison of PSD (laser diffraction)
and PSD (TEM) values of Sample C7.

| | PSD (laser diffraction) | | |
|---|---|---|---|
| | Dᵥ10-Dᵥ50-Dᵥ90 (nm); | PSD (TEM) | |
| | SSA (m²/g) | number distribution | E(1,1) |
| Material C7 | 204-492-2100; 14.3 | D_{N}10: 152.2 nm | 211 |
| | | D_{N}50: 277.4 nm | |
| | | Particles < 100 nm: | |
| | | 2.8% | |

TEM reveals that the particles in sample C7 were crystals, agglomerates were not detected. Nanoparticles are almost absent, this corresponds with the strong increase of Dv10 value obtained from laser diffraction.

This demonstrates that the fraction of nanoparticles <100 nm is reduced by use of the process according to the present invention. Non-nano UV Filter dispersions with good performance are accessible.

TABLE 6
Addition of Cetiol B (Dibutyl adipat);
PSD results are volume-based.

| | | | Laser diffraction |
|---|---|---|---|
| | wt-% of added oil | | Dᵥ10-Dᵥ50-Dᵥ90 (nm); SSA |
| No. | to C1 | T(° C.) | (m²/g) |
| C8 | 0.5% Cetiol B | 54 | 3 h: 102-280-1620; 27.3 |
| | | | 5 h 30: 148-339-1330; 20.5 |
| | | | 6 h 30: 159-349-1280; 19.5 |
| C9 | 0.5% Cetiol B | 31 | 4 h: 96-250-1217; 30.3 |
| | | | 8 h: 88-219-770; 34.6 |
| | temperature raised | 49-50 | 11 h 30: 83-201- 541; 37.5 |
| | after 8 h | | |
| C10 | 0.5% Cetiol B | 52 | 4 h: 152-345-1338; 20 |
| | | | 8 h: 184-368-1196; 17.7 |
| | temperature lowered | 35 | 11 h 30: 150-304-912; 22 |
| | after 8 h | | |

Cetiol B has a similar effect as Cetiol AB at elevated temperatures 52-54° C.

### Examples According to the Invention D1-D4

Oil was added to MBBT suspensions containing 50% of MBBT, water and polyglyceryl laurate. The suspension was micronized using 2 mm glass beads (in examples D1, D2) and 0.6-0.8 mm Y-stabilized ZrO2 beads (in examples D3, D4). Particle size was measured before oil addition and after the time indicated in the table. In example D1, the temperature of the suspension was increased after taking the first sample.

TABLE 7
Milling of UV Absorber 1 at different temperatures and
oil concentrations; PSD results are volume-based.

| | | | Laser diffraction |
|---|---|---|---|
| | wt-% of added oil | T | Dᵥ10- Dᵥ50- Dᵥ90 (nm); SSA |
| No. | to C0 | (° C.) | (m²/g) |
| D1 | 10% polyglyceryl laurate | 30 | start 76-178-834; 41.2 |
| | and 0.3% Cetiol B | 61 | 2 h 30: 161-324-1030; 20.4 |
| D2 | 10% polyglyceryl laurate | 56 | start: 78-191-1414; 38.5 |
| | and 0.4% Cetiol AB | 56 | 3 h 15: 134-286-1037; 24.2 |
| D3 | 8.5% polyglyceryl laurate | 46 | start: 85-210-697; 36.0 |
| | and 0.6% Cetiol AB | 48 | 3 h 50: 111-249-625; 29.1 |
| D4 | 8.5% polyglyceryl laurate | 48 | start: 97-252-1200 30.1 |
| | and 0.6% Cetiol AB | 49 | 1 h 10: 145-318-1200; 21.5 |

Milling of MBBT in polyglyceryl laurate solution with oil at elevated temperatures has a similar effect on PSD as in example C7.

### Examples According to the Invention D5-D6

Dispersions containing 50% of UV Absorber 2, decaglyceryl monolaurate and oil were micronized using 0.6-0.8 mm Y—ZrO2 beads at different conditions

TABLE 8
Milling of UV Absorber 2 in polyglyceryl laurate solution
with added oil at different temperatures and oil concentrations;
PSD results are volume-based.

| | | | Laser diffraction |
|---|---|---|---|
| | UV Absorber 2 | T | Dv10- Dv50- Dv90 (nm); SSA |
| No. | suspension | (° C.) | (m²/g) |
| D5 | 11% polyglyceryl | 49 | start: 126-1600-4000; 14.3 |
| | laurate and 0.6% | 53 | 1 h 00: 96-807-2010; 22.8 |
| | Cetiol AB | 52 | 2 h 00: 240-657-1900; 20.4 |
| D6 | 11% polyglyceryl | 46 | start: 97-888-2200; 21.8 |
| | laurate and 0.3% | 47 | 1 h 00:202-450-1800; 15.1 |
| | Cetiol AB | 47 | 1 h 55: 143-297-1300; 22.5 |
| | | 47 | 2 h 20: 126-267-787; 26.1 |

Milling of TBPT in polyglyceryl laurate solution with added oil at elevated temperatures has a similar effect on PSD as in example C7.

### Shelf Life Stability

The product obtained in Example C7 was stored at 40° C. for 5 weeks, and particle size was measured using laser light diffraction.
PSD, initial: Dᵥ10-Dᵥ50-Dᵥ90 (nm): 159-349-1280; SSA (m²/ g): 19.5
PSD, 5 wks@40° C.: Dᵥ10-Dᵥ50-Dᵥ90 (nm): 169-366-1250; SSA (m²/g): 18.4

1.-15. (canceled)

16. A process of manufacturing an aqueous suspension (1) of at least one organic UV filter having a particle size D_{N}30 of 100 nm or more, said process comprising the step of milling a suspension (2) comprising the at least one organic UV filter in a mixture of water and a hydrophobic additive in a milling apparatus at a temperature of 35 to 90° C.

17. The process according to claim 16, wherein the at least one organic UV filter in the aqueous suspension (1) has a particle size D_{N}10 of 100 nm or more; and/or
a particle size D_{N}50 of 120 nm or more; and/or
a particle size D_{N}90 of 200 nm or more; and/or
a particle size Dv10 determined by laser diffraction of more than 0.1 μm; and/or
a particle size Dv90 determined by laser diffraction of less than 2.2 μm,
where Dv10 and Dv90 are measured using a Mastersizer 2000 from Malvern Panalytical

18. The process according to claim 16, wherein the population of particles below 100 nm in the aqueous suspension (1) is reduced during the milling step.

19. The process according to claim 16, wherein the temperature in the milling step is in the range of 40 to 80° C.

20. The process according to claim 16, wherein the hydrophobic additive is a cosmetic oil selected from the group consisting of alcohols having from 6 to 18 carbon atoms; C6-C24 carboxylic acids; esters of C6-C24 carboxylic acids with C3-C24 alcohols; esters of hydroxycarboxylic acids with C6-C24 alcohols; esters of carboxylic acids with polyhydric alcohols; liquid mono-/di-/tri-glyceride mixtures based on C6-C18 carboxylic acids; esters of C6-C24 alcohols with aromatic carboxylic acids or with oxo carboxylic acids; tricarboxylic acid esters; esters of C2-C12 dicarboxylic acids with alcohols having from 1 to 22 carbon atoms or polyols having from 2 to 10 carbon atoms and from 2 to 6 hydroxy groups; substituted cyclohexanes; C6-C22 alcohol carbonates; symmetric or asymmetric dialkyl ethers having a total of from 12 to 36 carbon atoms; ring-opening products of epoxidized carboxylic acid esters with polyols; silicone oils; aliphatic or naphthenic hydrocarbons; diol esters; and mixtures thereof.

21. The process according to claim 16, wherein the at least one organic UV filter is an insoluble organic UV filter selected from the group consisting of oxanilide UV filter, triazine UV filter, piperazine UV filter, triazole UV filter, vinyl group-containing amide UV filter, cinnamic acid amide UV filter, sulfonated benzimidazole, and mixtures thereof, or oxanilide UV filter having the formula (1) 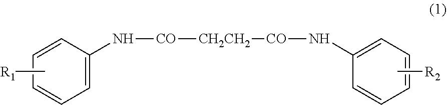
in which R₁ and R₂ are independently C1-C18 alkyl or C1-C18 alkoxy; triazine UV filter having the formula (2) 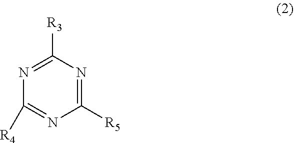
in which R₃, R₄ and R₅, independently, are H; OH; C1-C18 alkoxy; NH2; NH—R6 or N(R6)2 in which R6 is C1-C18 alkyl; OR6 in which R6 has its previous significance; phenyl; phenoxy; anilino; pyrrolo, in which the respective phenyl, phenoxy, anilino, or pyrrolo moieties are optionally substituted by one, two or three substituents selected from OH, carboxy, CO—NH2, C1-C18 alkyl or -alkoxy, C1-C18 carboxyalkyl, C5-C8 cycloalkyl, phenyl, a methylidenecamphor group, a group —(CH—CH)mC(═O)—OR6 in which m is 0 or 1 and R6 has its previous significance, or
a group 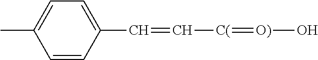
or the corresponding alkali metal, ammonium, mono-, di- or tri-C1-C4 alkylammonium, mono-, di- or tri-C2-C4 alkanolammonium salts, or the C1-C18alkyl esters thereof; phenylene bis-diphenyltriazine; a piperazine UV filter; a triazole UV filter having the formulae (31), (32) or (33) 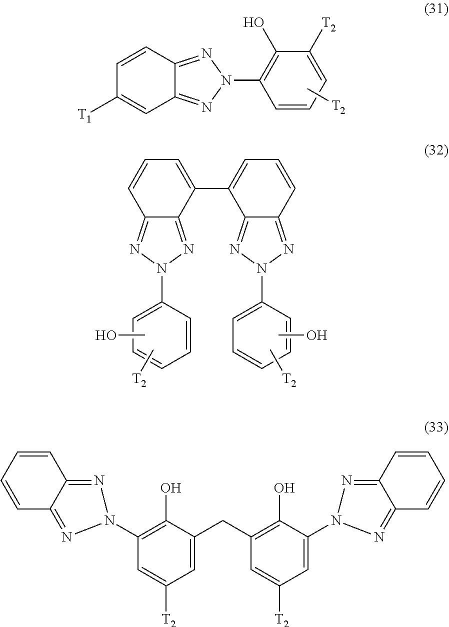
in which T₁ is C1-C18 alkyl or hydrogen and T₂ is C1-C18-alkyl, which is optionally substituted by phenyl; a vinyl group-containing amide UV filter having the formula (4)
R₉—(Y)ₘ——CO—C(R₁₀)═C(R₁₁)—N(R₁₂)(R₁₃) (4)
in which R₉ is C1-C18 alkyl or phenyl optionally substituted by one, two or three substituents selected from OH, C1-C18 alkyl, C1-C18 alkoxy or CO—OR6 in which R6 has its previous significance; R₁₀, R₁₁, R₁₂ and R₁₃ are the same or different and each is independently C1-C18 alkyl or hydrogen; Y is N or O; and m has its previous significance; cinnamic acid amide UV filter having the formula (5) 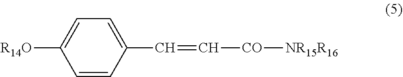
in which R₁₄ is hydroxy or C1-C4 alkoxy; R₁₅ is hydrogen or C1-C4 alkyl; and R₁₆ is —(CONH)m-phenyl in which m has its previous significance and the phenyl group is optionally substituted by one, two or three substituents selected from OH, C1-C18 alkyl, C1-C18 alkoxy or CO—OR6 in which R6 has its previous significance; sulfonated benzimidazole UV filter having the formula (6) 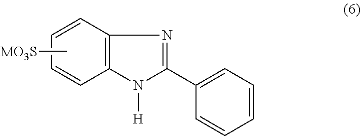
in which M is hydrogen or an alkali metal, an alkaline earth metal or zinc; and mixtures thereof.

22. The process according to claim 16, wherein the hydrophobic additive is comprised in the aqueous suspension (1) in an amount of 0.01 to 10.0 wt.-%, based on the total amount of the aqueous suspension (1).

23. The process according to claim 16, wherein the milling step is conducted in a ball mill, a vibratory mill, a wet rotor mill, a stirred media mill, or a colloid mill.

24. The process according to claim 16, wherein the at least one organic UV filter in suspension (2) has a particle size Dv90 determined by laser diffraction in the range of from 0.01 to 300 μm.

25. An aqueous suspension (1) comprising
a) 10 to 65 wt.-% of at least one organic UV filter having a particle size D_{N}30 of 100 nm or more
b) 0.01 to 10.0 wt.-% of a hydrophobic additive selected from the group consisting of alcohols having from 6 to 18 carbon atoms; C6-C24 carboxylic acids; esters of C6-C24 carboxylic acids with C3-C24 alcohols; esters of hydroxycarboxylic acids with C6-C24 alcohols; esters of carboxylic acids with polyhydric alcohols; liquid mono-/di-/tri-glyceride mixtures based on C6-C18 carboxylic acids; esters of C6-C24 alcohols with aromatic carboxylic acids or with oxo carboxylic acids; tricarboxylic acid esters; esters of C2-C12 dicarboxylic acids with alcohols having from 1 to 22 carbon atoms or polyols having from 2 to 10 carbon atoms and from 2 to 6 hydroxy groups; substituted cyclohexanes; C6-C22 alcohol carbonates; symmetric or asymmetric dialkyl ethers having a total of from 12 to 36 carbon atoms; ring-opening products of epoxidized carboxylic acid esters with polyols; silicone oils; aliphatic or naphthenic hydrocarbons; diol esters; and mixtures thereof, and
c) water
each based on the total amount of the aqueous suspension (1).

26. The aqueous suspension (1) according to claim 25, wherein the hydrophobic additive is selected from the group consisting of esters of C6-C24 alcohols.

27. The aqueous suspension (1) according to claim 25, wherein the at least one organic UV filter is an insoluble organic UV filter, selected from the group consisting of oxanilide UV filter, triazine UV filter, piperazine UV filter, triazole UV filter, vinyl group-containing amide UV filter, cinnamic acid amide UV filter, sulfonated benzimidazole, and mixtures thereof, or selected from the group consisting of oxanilide UV filter having the formula (1) 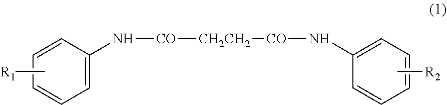
in which R₁ and R₂ are independently C1-C18 alkyl or C1-C18 alkoxy; triazine UV filter having the formula (2) 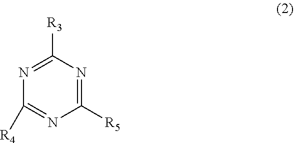
in which R₃, R₄ and R₅, independently, are H; OH; C1-C18 alkoxy; NH2; NH—R6 or N(R6)2 in which R6 is C1-C18 alkyl; OR6 in which R6 has its previous significance; phenyl; phenoxy; anilino; pyrrolo, in which the respective phenyl, phenoxy, anilino, or pyrrolo moieties are optionally substituted by one, two or three substituents selected from OH, carboxy, CO—NH2, C1-C18 alkyl or -alkoxy, C1-C18 carboxyalkyl, C5-C8 cycloalkyl, phenyl, a methylidenecamphor group, a group —(CH═CH)mC(—O)—OR6 in which m is 0 or 1 and R6 has its previous significance, or
a group 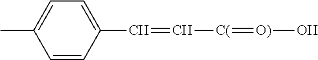
or the corresponding alkali metal, ammonium, mono-, di- or tri-C1-C4 alkylammonium, mono-, di- or tri-C2-C4 alkanolammonium salts, or the C1-C18alkyl esters thereof, phenylene bis-diphenyltriazine; a piperazine UV filter; a triazole UV filter having the formulae (31), (32) or (33) 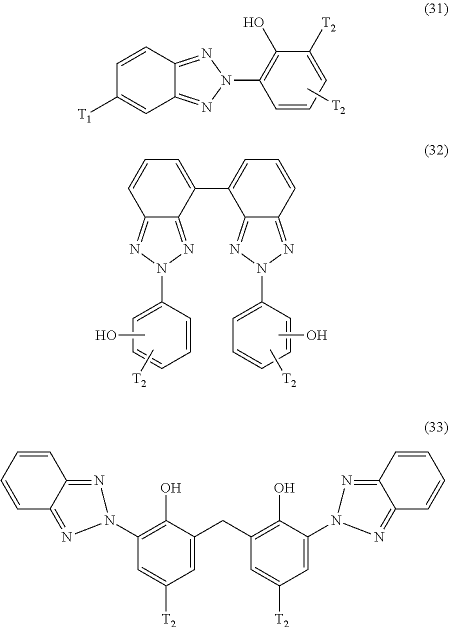
in which T₁ is C1-C18 alkyl or hydrogen and T₂ is C1-C18-alkyl, which is optionally substituted by phenyl; a vinyl group-containing amide UV filter having the formula (4)
R₉—(Y)ₘ—CO—C(R₁)═C(R₁₀)—N(R₁₂)(R₁₃) (4)
in which R₉ is C1-C18 alkyl or phenyl optionally substituted by one, two or three substituents selected from OH, C1-C18 alkyl, C1-C18 alkoxy or CO—OR6 in which R6 has its previous significance; R₁₀, R₁₁, R₁₂ and R₁₃ are the same or different and each is independently C1-C18 alkyl or hydrogen; Y is N or O; and m has its previous significance; cinnamic acid amide UV filter having the formula (5) 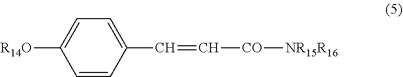
in which R₁₄ is hydroxy or C1-C4 alkoxy; R₁₅ is hydrogen or C1-C4 alkyl; and R₁₆ is —(CONH)m-phenyl in which m has its previous significance and the phenyl group is optionally substituted by one, two or three substituents selected from OH, C1-C18 alkyl, C1-C18 alkoxy or CO—OR6 in which R6 has its previous significance; sulfonated benzimidazole UV filter having the formula (6) 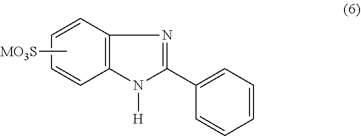
in which M is hydrogen or an alkali metal, an alkaline earth metal or zinc; and mixtures thereof.

28. The aqueous suspension (1) according to claim 25, wherein the aqueous suspension (1) comprises the hydrophobic additive in an amount of 0.01 to 8.0 wt.-%, based on the total amount of the aqueous suspension (1).

29. The aqueous suspension (1) according to claim 25, wherein the at least one organic UV filter has a particle size D_{N}10 of 100 nm or more; and/or
a particle size D_{N}50 of 120 nm or more; and/or
a particle size D_{N}90 of 200 nm or more; and/or
a particle size Dv10 determined by laser diffraction of more than 0.1 μm; and/or
a particle size Dv90 determined by laser diffraction of less than 2.2 μm,
where Dv10 and Dv90 are measured using a Mastersizer 2000 from Malvern Panalytical.

30. The aqueous suspension (1) according to claim 25 for use in sunscreen or daily care composition.
